(19) United States
(12) Patent Application Publication
(10) Pub. No.: US 2026/0060635 A1
(43) Pub. Date: Mar. 5, 2026

(54) EXPLAINABLE DEEP LEARNING METHOD FOR NON-INVASIVE DETECTION OF PULMONARY HYPERTENSION FROM HEART SOUNDS

(71) Applicants: INESC TEC - INSTITUTO DE ENGENHARIA DE SISTEMAS E COMPUTADORES, TECNOLOGIA E CIÊNCIA, Porto (PT); UNIVERSIDADE DO PORTO, Porto (PT); AALBORG UNIVERSITET, Aalborg (DK)

(72) Inventors: Alex GAUDIO, Mystic, CT (US); Francesco RENNA, Porto (PT); Samuel SCHMIDT, AALBORG SØ (DK); Miguel TAVARES COIMBRA, Porto (PT)

(73) Assignees: INESC TEC - INSTITUTO DE ENGENHARIA DE SISTEMAS E COMPUTADORES, TECNOLOGIA E CIÊNCIA, Porto (PT); UNIVERSIDADE DO PORTO, Porto (PT); AALBORG UNIVERSITET, Aalborg (DK)

(21) Appl. No.: 19/108,279

(22) PCT Filed: Sep. 1, 2023
(86) PCT No.: PCT/IB2023/058675

§ 371 Date: Mar. 3, 2025

(30) Foreign Application Priority Data
Sep. 2, 2022 (PT) 118182

(51) Int. Cl. A61B 7/00; A61B 5/00; G16H 50/20

(52) U.S. Cl. CPC A61B 7/003; A61B 5/7267; G16H 50/20

(57) ABSTRACT

Disclosed is a computer-implemented method for non-invasive estimation of Pulmonary Hypertension, PH, from heart sound signals. Consistent with the disclosure, the method includes the steps of: receiving a sound signal acquired from a beating heart of a subject over a predetermined time period; generating one or more 2D feature maps comprising a 2D feature map with the received sound signal where a first axis of the map is arranged over time and a second axis of the map is arranged over individual heartbeats; applying a pre-trained neural network to relate the generated one or more 2D feature maps with a training dataset of previously acquired and generated training 2D feature maps of a PH subject group and a non-PH subject group, thus to obtain an indicator of the presence of Pulmonary Hypertension. Also disclosed is a training method of said neural network and a system.

### TECHNICAL FIELD

The present disclosure relates to an explainable deep learning method for non-invasive detection of pulmonary hypertension from heart sounds.

### BACKGROUND

Pulmonary Hypertension (PH) is an underrecognized disease, with unmet need for diagnostic and treatment recommendations in low and middle-income regions [1]. PH disease has high mortality rate and early detection in screening programs can improve outcomes. Existing tools for PH detection are not well optimized for the needs of low- and middle-income regions.

Right heart catheterization is a gold standard for PH detection, but it is very costly, highly invasive, not suitable for screening programs and requires a specialized team of well-trained clinicians.

Implantable pressure sensors are placed in the pulmonary artery, these are extremely costly and highly invasive. It gives the best blood pressure readings, but only applies to a very small portion of subjects in wealthy countries.

Doppler echocardiography is widely used for clinical screening of PH, but the noisy nature of its measurements requires additional modalities to improve reliability [2, 3]. It only gives pulmonary pressure predictions in some subjects. Ultrasound technology also requires a trained technician and expensive machinery [4].

Other tests helpful to PH detection include blood gas analysis and imaging from cardiac magnetic resonance, chest x-ray, and pulmonary angiography [5]. Several constraints limit applicability of these tests, including low predictive performance, lack of explainability, higher cost, or more invasive nature of the tests. Moreover, these tests can support PH detection but are not definitive tests for PH detection. Automated PH detection using cardiac auscultation data recently emerged as a non-invasive and low-cost alternative that can outperform physicians [6], however these tests also constrained by low predictive performance, lack of explainability, and a requirement that they use synchronized electrocardiogram (EKG) alongside heart sound signals.

Detection of PH from heart sounds focuses on an analysis of the second heart sound, S2, which itself consists of two mixed sound signals: the louder Aortic valve closure (A2) and the quieter Pulmonic valve closure (P2) [7]. Peak-to-peak analysis, in the time domain, shows that subjects with PH disease present with larger distance and larger difference in amplitude between the A2 and P2 peaks [6].

Automated diagnosis of PH from heart sound includes handcrafted analysis [8] and traditional machine learning [6, 9]. In a related area, application of deep Convolutional Neural Networks (CNNs) is useful in heart murmur detection in children [4] and heart sound segmentation [10].

Existing automated methods to estimate PH or PAP from heart sounds do not provide sufficiently accurate results.

The known deep learning methods do not explain which part of the heart sounds are responsible for the output.

Poor subject outcomes as a result of late diagnoses of pulmonary hypertension (PH) highlights the need for an earlier, non-invasive PH detection. Cardiac auscultation offers a non-invasive and cost-effective alternative to right heart catheterization, CardioMEMS, and doppler analysis in analysis of PH, however it represents an indirect measurement of the pulmonary pressure, therefore, it needs to be properly validated in different scenarios.

These facts are disclosed in order to illustrate the technical problem addressed by the present disclosure.

### GENERAL DESCRIPTION

The present document proposes to detect Pulmonary Hypertension (PH) via the analysis of digital heart sound recordings with over-parameterized deep neural networks. It is further disclosed a pre-processing step aiming to separate S2 sound into the aortic (A2) and pulmonary (P2) components, and an explanation of the prediction. It is also disclosed a deep neural network architecture, optional compression step, and optional alternative training method that yields a highly accurate and low resource requirement predictive model.

It was obtained an area under the ROC curve of 0.95, improving over the state-of-the-art Gaussian mixture model PH detector by 0.17. Post-hoc explanations and analysis show that the availability of separated A2 and P2 components contributes significantly to prediction.

Analysis of stethoscope heart sound recordings with deep networks is an effective, low-cost, and non-invasive solution for the detection of pulmonary hypertension.

This approach adopts deep convolutional neural networks (CNNs) [11, 12], typically used for image analysis, for the analysis of audio data. This approach also adopts post-hoc attribution methods like Integrated Gradients [13], typically applied to CNN outputs, to develop an explanation of PH detection on the subject's overall audio signal data and of individual heartbeats.

It is known that deep networks typically require large training datasets, and that Gaussian Mixture Model (GMM) and Support Vector Machines (SVMs), previous state-of-the-art methods, do not scale to large datasets. So, one of the novel aspects of the present disclosure on the PH detection is to propose deep networks using datasets of any size, with specific optimizations to use deep networks on small data.

These optimizations comprise:
alternative training mechanism based on fixed-weight neural networks and/or non-iterative (i.e. one training step) optimization;
alternatively batch gradient descent instead of minibatch gradient descent;
optionally zero padding heartbeats to give all subjects the same number of heartbeats, i.e., passing a same size image to the CNN;
optionally channel normalization to stabilize gradient backpropagation via equation:

$$( x ) / {channel}_{std} ( x )$$

where x is a “colour channel”, of the 3-channel input passed to CNN.

It is disclosed that: applying deep networks to analysis of heart sound recordings gives strong predictive performance; and post-hoc explanations verify the role of proposed A2 and P2 components in the second heart sound.

In an embodiment, the method uses physiologically relevant features that correspond to at least one domain knowledge, preferably the physiologically relevant features are the characteristics of the P2 components and its relationship with respect to the A2 components.

Advantages of the disclosed explanation method include:
enhancing trustworthiness of model for a given subject by verifying (a) the model behaves according to domain knowledge and (b) the prediction is not unlike other predictions by this model. Explanations enhance trustworthiness and are essential for decision making.
per-heartbeat explanations of region of interest across time, and also across channel, i.e., proposed A2, proposed P2, S2;
aggregated per-subject explanations of region of interest across time and channel. Thus, one can aggregate the per-heartbeat explanations to give an overall impression of prediction in context of the subject.

The present document discloses a computer-implemented method for non-invasive estimation of Pulmonary Hypertension, PH, from heart sound signals, comprising the steps:
receiving a sound signal (S2) acquired from a beating heart of a subject over a predetermined time period;
generating one or more 2D feature maps comprising a 2D feature map with the received sound signal (S2) where a first axis of the map is arranged over time and a second axis of the map is arranged over individual heartbeats;
applying a pre-trained neural network to relate the generated one or more 2D feature maps with a training dataset of previously acquired, and generated training 2D feature maps of a PH subject group and a non-PH subject group, in order to obtain an indicator of the presence of Pulmonary Hypertension.

This method, with a single input channel (i.e., no splitting), is, at least, as accurate, significantly faster and has lower resource usage than the following method with the extra step of splitting the sound signal (S2) into proposed A2 and P2 signals.

While deep learning approaches are almost always updated by backpropagation, in an embodiment the method has no backpropagation, being the network “wide” rather than “deep”.

Optionally, the method incorporates a dimensionality reduction, e.g., a Principal Component Analysis (PCA), into the deep network architecture, and serial processing of parallel convolution blocks that enables the RAM usage to be adjustable for a given device. So, it can be evaluated and trained on a mobile device, e.g., laptop.

In an alternative embodiment, it is also disclosed a computer-implemented method for non-invasive estimation of Pulmonary Hypertension, PH, from heart sound signals, comprising the steps:
receiving a sound signal (S2) acquired from a beating heart of a subject over a predetermined time period;
splitting the sound signal (S2) into an aortic sound signal (A2) and a pulmonary sound signal (P2);
generating one or more 2D feature maps comprising a 2D pulmonary feature map with the pulmonary sound signal (P2) where a first axis of the map is arranged over time and a second axis of the map is arranged over individual heartbeats;
applying a pre-trained neural network to relate the generated one or more 2D feature maps with a training dataset of previously acquired, split and generated training 2D feature maps of a PH subject group and a non-PH subject group, thus to obtain an indicator of the presence of Pulmonary Hypertension.

In an embodiment, the one or more 2D feature maps comprising a 2D aortic feature map with the aortic sound signal (A2), where a first axis of the map is arranged over time and a second axis of the map is arranged over individual heartbeats.

In an embodiment, the one or more 2D feature maps comprising a 2D full-signal feature map with the received sound signal (S2), where a first axis of the map is arranged over time and a second axis of the map is arranged over individual heartbeats.

In an embodiment, said method further comprising combining the one or more 2D feature maps as a multichannel input to the neural network, where each one or more 2D feature maps is combined as a channel of the multichannel input.

In an embodiment, said method further comprising a multi-channel input for a neural network, where for the case of three feature maps, each channel is considered a colour channel of a generated image, and where for the case of one feature map is considered as a grayscale image.

In an embodiment, said method further comprising an image for a neural network, where the one or more feature maps are combined as colour channels of the generated images.

In an embodiment, said method comprising segmenting the acquired sound signal into a plurality of time windows of a predetermined duration, each time window comprising a heartbeat sound signal peak, preferably predetermined duration being 200 milliseconds.

In an embodiment, said method comprising aligning the segmented sound signal time windows by aligning the heartbeat sound signal peaks of the segmented sound signal time windows.

In an embodiment, said method further comprising calculating saliency attribution, preferably via integrated gradients or gradient times corresponding to the generated one or more 2D feature maps.

In an embodiment, said method comprising pre-processing the acquired sound signal by filtering, spike removal, normalizing, alignment, or segmentation, or a combination thereof.

In an embodiment, wherein the neural network is a convolutional neural network, CNN.

In an embodiment, wherein the neural network is an over-parameterized deep neural network.

In an embodiment, wherein the neural network is an extreme learning machine.

In an embodiment, wherein the neural network is a fixed-weight deep or wide neural network. By fixed-weight means that the weights are not modified by optimization (not modified by training).

In an embodiment, wherein the splitting is performed using an alternating optimization of a least-squares problem.

In an embodiment, said method further comprising, after splitting the heart sound signal, filtering with second order Butterworth filters, in particular with Butterworth filters with cut-off frequencies of 25 Hz and 400 Hz, re-sampling to 1 kHz, and cleaning by removing spikes.

In an embodiment, said method comprising acquiring the heart sound signal at the subject's pulmonary spot, preferably over the second left intercostal space.

It is also disclosed a computer-implemented method for training neural network for a non-invasive estimation of Pulmonary Hypertension, PH, from heart sound signals, comprising the steps, for both of a PH subject group and a non-PH subject group:
receiving a sound signal (S2) acquired from a beating heart of a subject over a predetermined time period;
splitting the heart sound (S2) signal into an aortic (A2) sound signal and a pulmonary (P2) sound signal;
generating one or more 2D feature maps comprising a 2D pulmonary feature map with the pulmonary sound signal (P2) where a first axis of the map is arranged over time and a second axis of the map is arranged over individual heartbeats;
applying a pre-trained neural network to relate the generated one or more 2D feature maps with a training dataset of previously acquired, split and generated training 2D feature maps of a PH subject group and a non-PH subject group, and to obtain an indicator of the presence of Pulmonary Hypertension.

It is further disclosed a computer-implemented system for non-invasive estimation of Pulmonary Hypertension, PH, from heart sound signals, comprising an electronic data processor arranged to carry out the steps:
receiving a sound signal (S2) acquired from a beating heart of a subject over a predetermined time period;
splitting the sound signal (S2) into an aortic sound signal (A2) and a pulmonary sound signal (P2);
generating one or more 2D feature maps comprising a 2D pulmonary feature map with the pulmonary sound signal (P2) where a first axis of the map is arranged over time and a second axis of the map is arranged over individual heartbeats;
applying a pre-trained neural network to relate the generated one or more 2D feature maps with a training dataset of previously acquired, split and generated training 2D feature maps of a PH subject group and a non-PH subject group, and to obtain an indicator of the presence of Pulmonary Hypertension.

In an embodiment, said system comprising a digital stethoscope for acquiring the beating heart sound signal, wherein the digital stethoscope is connected to the electronic data processor for transmitting the acquired beating heart sound signal.

In an embodiment, the electronic data processor is further arranged to segment the acquired sound signal into a plurality of time windows of a predetermined duration, each time window comprising a heartbeat sound signal peak, preferably the predetermined duration being 200 milliseconds.

In an embodiment, the electronic data processor is further arranged to align the segmented sound signal time windows by aligning the heartbeat sound signal peaks of the segmented sound signal time windows.

In an embodiment, the electronic data processor is embodied on a mobile device.

### BRIEF DESCRIPTION OF THE DRAWINGS

The following figures provide preferred embodiments for illustrating the disclosure and should not be seen as limiting the scope of invention.

FIG. 1: Graphical representation of an S2 audio signal, a proposed A2 audio signal, a proposed P2 audio signal, and an average feature attribution over time.

FIGS. 2A, 2B, 2C: Graphical representation of an embodiment of a subject audio data and a respective explanation, visualized as 3-channel images.

FIG. 3: Graphical representation comprising the ROC Curve for the disclosed method, herein named DeepPHDet, a GMM, and a SVM model. demonstrating superior predictive performance of DeepPHDet over existing state-of-the-art baselines.

FIG. 4: Flowchart representation of an embodiment of a method for non-invasive estimation of Pulmonary Hypertension, PH, from heart sound signals.

### DETAILED DESCRIPTION

It is disclosed an algorithm for non-invasive detection of pulmonary hypertension (PH). Heart sounds are collected from subjects with a digital stethoscope, and subsequently passed as audio input to the said algorithm. The output is an estimate of PH as well as an explanation of relevant regions of the subject's heart sounds. The heart sound audio recording is collected from the subject's pulmonary spot, e.g., on the left hand side of the sternum, in the second intercostal space. The algorithm has a training phase, in which it gains domain knowledge from multiple subject recordings, and an evaluation phase, in which it makes a prediction for individual subjects.

In an embodiment, the predictions are made over subjects that were not considered in the training phase.

In an embodiment, the algorithm begins with a set of pre-processing steps that include filtering, spike removal, and segmentation of S2 sounds from heart sound recordings. Then, a source separation algorithm is applied to the S2 sounds of a recording in order to separate each S2 sound into its aortic (A2) and pulmonary (P2) components. The obtained S2 sounds, together with the corresponding A2 and P2 components, are organized and normalized into feature maps of the same shape as 3-channel images that are provided as input of a deep neural network. During training, several steps were taken to ensure the algorithm works with small data, including use of batch gradient descent, channel normalization to unit variance, and zero padding the input to ensure all inputs have the same number of heartbeats. At evaluation, the algorithm predicts whether a subject has PH or not. In addition, the components of the heart sounds, moments in time, and heartbeats that are most informative to the prediction are highlighted via a post-hoc explanation attribution method and aggregation method.

Alternatively, either one of two optimization approaches for training the network are applied to ensure the algorithm works with small data. The first optimization approach makes use of the following steps: use of batch gradient descent rather than minibatch or stochastic gradient descent (the parameter update step occurs once per iteration of the entire data set, but the gradients may be accumulated using minibatches), channel normalization to unit variance, and in some cases, zero padding or cropping the input to ensure all inputs have the same number of segmented heartbeats. The second approach also employs the following steps: the neural network is assigned fixed parameter weights that are not updated by training, and the optimization method trains a final classifier layer or model using an analytically derived solution; sparsity regularization may be employed; compression may be employed.

Architectural choices were also made to ensure the model works efficiently with the dataset, including: choice of the convolution layer architecture to compute parallel convolutions in series to reduce RAM use, choosing a kernel width large enough to match the sampling rate of the sound signal, and careful use of pooling to perform a mathematical transformation of the input signal into a compressible latent space, and a compression method to reduce the size of the embedding space.

The model's prediction depends primarily on the proposed P2 heart sound, especially in the region of most variation at 30 ms to 60 ms.

TABLE 1
The dataset summary.

| Population | Male | Female | Age | HR (bpm) |
|---|---|---|---|---|
| Has PH | 9 | 20 | 60 ± 17 | 70 ± 10 |
| No PH | 8 | 5 | 57 ± 10 | 69 ± 8 |
| All subjects | 17 | 25 | 59 ± 15 | 69 ± 9 |

It was acquired a private dataset of 42 subjects at Centro Hospitalar Universitário do Porto, Portugal. Summary statistics in Table 1 show 29 subjects with PH and 13 without PH. Of diseased subjects, the majority, 20 of 29, are female. The age and heart rates of both positive and diseased populations are similar. Inclusion and exclusion criteria are unknown. PH is defined as positive when a subject has a Mean Pulmonary Arterial Pressure (MPAP) above 25 mm Hg, or Pulmonary Arterial Systolic Pressure (PASP) above 30 mm Hg. For each subject, it was obtained the ground truth pulmonary artery pressure from a right heart catheterization, and an accompanying five-minute PCG heart sound recording. The recording was obtained in a relatively quiet clinical setting with the subject supine and at rest. Auscultation was performed over the second left intercostal space using a custom cable stethoscope connected to a Rugloop Waves® system. Heart sounds were recorded at a sample rate of 8 kHz and their amplitudes were quantized with 16-bit resolution. The dataset is not published to preserve privacy.

In each five-minute audio signal, the heartbeats were segmented and extracted into a 200 ms window for each heartbeat's S2 sound, where the start time of the window is chosen so the peaks of all S2 sounds for that subject are aligned in time. The S2 signal is filtered with second order Butterworth filters with cut-off frequencies of 25 Hz and 400 Hz, re-sampled to 1 kHz, cleaned by removing spikes via the method in [14], and separated into proposed A2 and P2 components according to [15]. Source separation assumes the Aortic and Pulmonic components maintain approximately the same waveform across heartbeats and assumes the delay between the components within a heartbeat varies due to change in thoracic pressure at different respiratory phases. The two components are retrieved via alternating optimization of a least-squares problem.

In an embodiment, the duration of each window of audio signal is a predefined parameter set by a user, e.g., a 200 ms for a sample rate of 1 kHz.

In an example, alignment and segmentation results in a multi-channel 2-D representation of the audio data containing S2, proposed A2, and proposed P2 components. Each 2-D channel has 200 columns, representing a 200 ms window, and as many rows as there are heartbeats. Then make channels for all subjects of the same shape by zero padding to 454 rows, and independently normalize each of the three channels per subject to unit variance. Normalizing to unit variance helps stabilize gradient back-propagation by reducing risk of vanishing or exploding gradients.

In an embodiment, it was considered DenseNet121, ResNet18 and EfficientNet-b0 architectures.

In an embodiment, pre-trained deep network initialization improves performance, in particular for small datasets.

In an embodiment, random and ImageNet initializations were considered.

It is disclosed a DenseNet trained from random initialization, ResNet18 from ImageNet initialization and EfficientNet-b0 from standard adversarial ImageNet initialization. The models were all trained with batch Gradient Descent, learning rate 0.0001, momentum 0.5, for 150 epochs. Deep networks typically train on large datasets with minibatch gradient descent. To stabilize gradient updates, it was performed a batch gradient descent, which means to perform a gradient update once per iteration over the dataset. To work with datasets of arbitrary size, we compute gradient updates once per sample and maintain a sum or running mean until a gradient update occurs. The loss is weighted binary cross entropy with the positive class balancing weight

$$\frac{8 + 5}{20 + 9} .$$

To benchmark the predictive performance of the deep networks against classical methods, it was implemented a Gaussian Mixture Model (GMM) and Support Vector Machine (SVM).

The present GMM implementation adapts the state-of-the-art work of [6], where one GMM was trained for positive classes, and another for negative classes. The class of a test sample is the GMM model with higher posterior negative log likelihood. To get best performance with this baseline, it was developed a different pre-processing pipeline, and accordingly optimized the GMM models to have two components and spherical covariance. The SVM uses an RBF kernel and slack parameter C=1. For pre-processing, it was used only the S2 channel. The addition of proposed A2 and P2 channels negatively impacts performance due to overfitting. Each of the heartbeats, each row of the S2 channel, was transformed with a 1-d Short Time Fourier Transform, using an FFT window of 64 samples and hop length of two samples, and computing the energy spectrum via absolute value. The subject data, a tensor of shape (H,33,101), was reduced to (33,101) by computing a 98% quantile over the H heartbeats. The channel was zero padded to 454 rows and normalized to unit variance, then flattened as a vector and subsequently passed to the SVM and GMM models.

All models were evaluated using 10-fold stratified cross validation. To report performance, it was stored a validation set prediction probabilities from each fold. There is one prediction probability for each subject. It is reported the area under the ROC curve (ROC AUC) and standard classification metrics. Classification metrics require choosing a threshold to convert the probabilities into classes. It was chosen a threshold Tk for each kth fold that maximizes the difference of true positive rate minus the false positive rate on the kth fold training set ROC curve. This threshold optimizes the training set balanced accuracy score. Validation performance was computed within each fold and then aggregate the metrics by an average across folds and epochs 100 to 150.

To better understand which parts of the proposed A2 and proposed P2 channels contribute to PH detection, it was applied the Integrated Gradients attribution method [13].

In an embodiment, after training the DenseNet121 model on ten folds, ten independently trained models are obtained. Therefore, ten attributions to each heartbeat in the dataset are computed and then averaged to get one attribution per channel or summed to get one importance score per heartbeat. For better visualization, the attribution is converted to a magnitude via absolute value and then clipped to 1% and 99% of its values. Clipping aids visualization because gradient-based attribution methods generate some outlier points.

TABLE 2
DeepPHDet* Gives State-of-the-art Results

| Model | ROC AUC | MCC | BAcc | Precision | Recall |
|---|---|---|---|---|---|
| GMM | 0.78 | 0.57 | 0.78 | 0.92 | 0.82 |
| SVM | 0.88 | 0.55 | 0.78 | 0.97 | 0.65 |
| DenseNet121 | 0.95 | 0.82 | 0.91 | 0.96 | 0.90 |
| (S2, A2, P2)* | | | | | |
| EfficientNet-b0 | 0.93 | 0.79 | 0.90 | 1.00 | 0.81 |
| (S2, A2, P2)* | | | | | |
| ResNet18 | 0.92 | 0.53 | 0.77 | 0.88 | 0.59 |
| (S2, A2, P2)* | | | | | |
| DenseNet121 (S2) | 0.93 | 0.69 | 0.85 | 0.94 | 0.81 |
| EfficientNet-b0 (S2) | 0.89 | 0.52 | 0.76 | 0.85 | 0.84 |

The results in Table 2 show that the DenseNet121 and EfficientNet-b0 deep networks outperform state-of-art machine learning models on the considered PH dataset by large margins. The DenseNet121 model has the highest performance of 0.95 ROC AUC, the highest Balanced Accuracy (BAcc), and highest Matthew's Correlation Coefficient (MCC). The two best performing models are DenseNet121 and EfficientNet-b0.

The bottom rows of Table 2 show that availability of S2, A2 and P2 channels improves performance over using only the S2. A motivation of deep learning is to overcome the need for pre-processing via data-driven feature generation and larger datasets. In the small data regime, as is the case here, it was observed that pre-processing improves performance. Moreover, the over-parameterized nature of deep networks required a rethinking from the state-of-art interpretations of underfitting and overfitting. Classical methods like the SVM and GMM overfit with additional parameters from the A2 and P2 channels while deep networks improve.

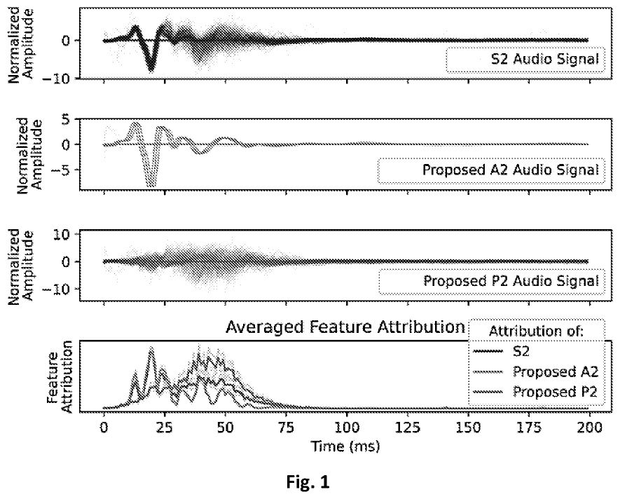

FIG. 1 shows a graphical representation of an S2 audio signal, a proposed A2 audio signal, a proposed P2 audio signal, and an average feature attribution over time.

The top three rows of FIG. 1 visualize one subject's heart sound data. Each line represents a single heartbeat. The top row shows the S2 signal. The second and third rows show the proposed source separated signals A2 and P2. The shown signals were normalized to unit variance to represent the input as passed to the predictive model.

It was found empirically that the normalization improved performance; normalization makes the quieter P2 have similar amplitude to the louder A2. The A2 signal is very clearly defined, due to the fact that the heartbeats have been aligned based on their peak. The distance between A2 and P2 components varies depending on factors such as whether the subject is inhaling or exhaling, as well as presence of PH. Thus, current domain knowledge agrees with the visual that an average P2 signal should be less well located in time. In this example, it was observed that the P2 has most varied behavior between 30 ms to 60 ms. Current domain knowledge expects PH to be related to changes in the timing and amplitude of the P2.

The bottom plot in FIG. 1 shows the average attribution over all heartbeats and a 99.9% confidence interval. The attribution to P2 dominates for this example, and also coincides with the period between 30 ms to 60 ms of most varied P2 behavior. Both observations suggest Deep Networks agree with domain knowledge. The attribution to A2 is strongest at the peak, just before 25 ms. Attribution shows the availability of separated components facilitates prediction.

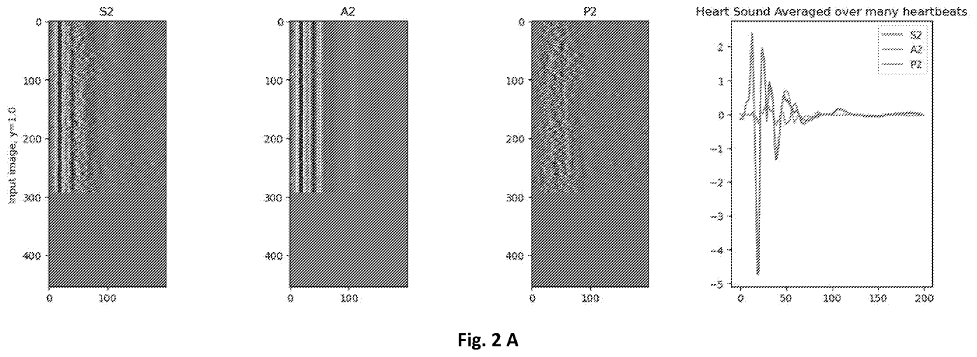

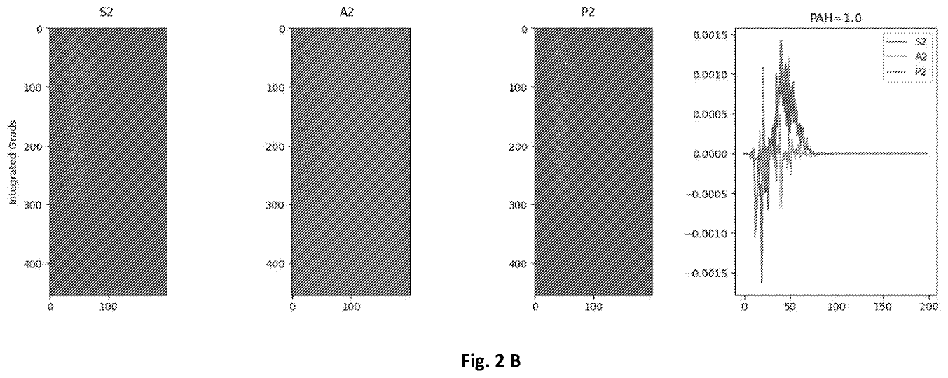

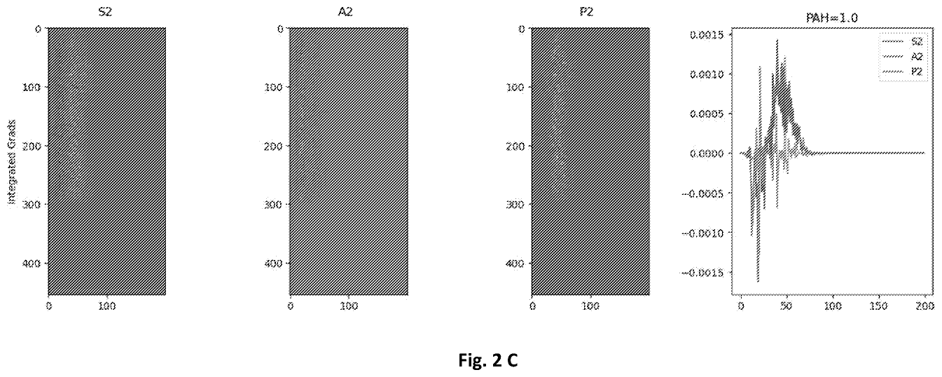

FIGS. 2A, 2B, 2C show a graphical representation of an embodiment of a subject audio data and a respective explanation, visualized as 3-channel images.

The first row is the input to a CNN, 2nd and third rows are outputs of attribution methods. The first three columns are the S2, Proposed A2 and Proposed P2 components. The fourth column represents an aggregated view of the waveforms across all heartbeats. In the plots, all heartbeats have been zero padded and the 2nd and 3rd rows use inputs that were normalized to unit variance before computing the attribution.

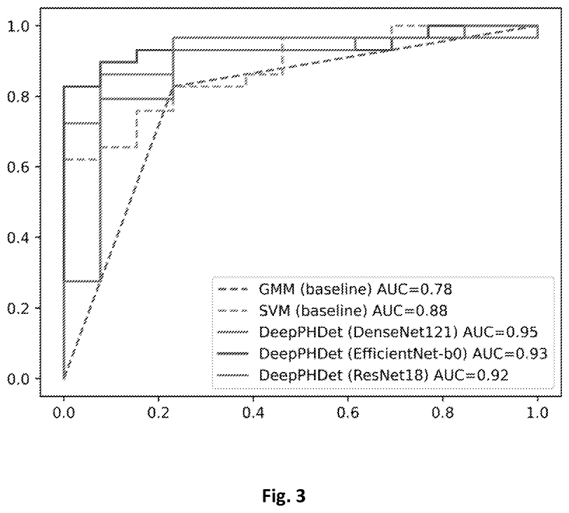

FIG. 3 shows a graphical representation comprising the ROC Curve for the disclosed method, herein named DeepPHDet, a GMM, and a SVM model. It is demonstrated superior predictive performance of DeepPHDet over existing state-of-the-art baselines.

It was found that deep networks improve detection performance; separating S2 into A2 and P2 may improve performance and improves explainability of the model and analysed S2 signal; the proposed A2 and P2 agree with domain knowledge; the post-hoc explanation validates domain knowledge and utility of A2 and P2 segmentations.

The present disclosure contributes, then, to the advance of the state-of-the-art in automated detection of pulmonary hypertension, namely pulmonary artery hypertension, from heart sounds. It comprises several advantages, such as: high predictive performance; suitability for training with small and large datasets; explanations of the A2 and S2 components that explain the prediction; and requires only heart sound data for inference.

It is shown that deep networks trained on a private dataset of pre-processed digital stethoscope recordings achieve ROC AUC scores of 0.95 and 0.93, giving improvements of +0.17 and +0.15 over an adaptation of a previous state-of-the-art based on a Gaussian Mixture Model, and improvements of +0.07 and +0.05 over state-of-art machine learning implementation.

Post-hoc explanations and improved performance show that the separation of the S2 sound into proposed A2 and P2 components aids detection.

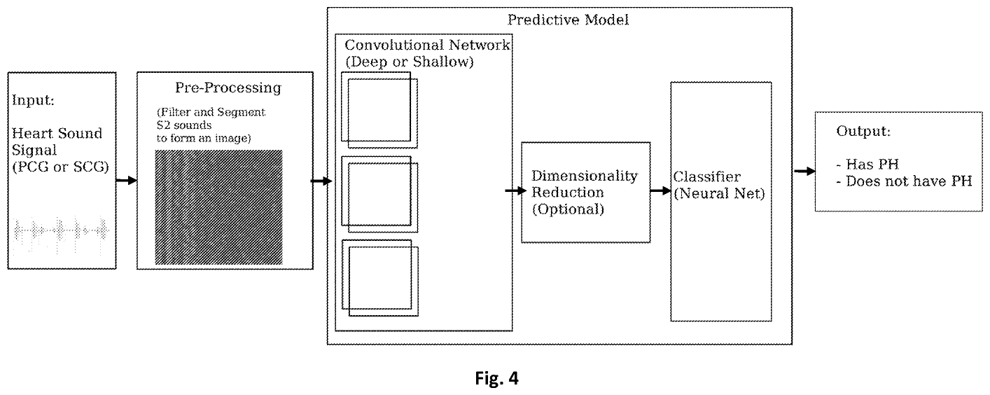

FIG. 4 shows a flowchart representation of an embodiment of a method for non-invasive estimation of Pulmonary Hypertension, PH, from heart sound signals.

In an embodiment, the whole model is trained via backpropagation.

In another embodiment, during training the convolutional network weights are initialized and fixed (never modified), and remaining steps are obtained by techniques of extreme learning machines or regression models.

Tests were performed on 3 datasets, obtained via stethoscope (PCG) and seismocardiogram (SCG) devices, containing recordings of humans and pigs. Namely, the Human Dataset, PCG:
42 human subjects undergoing right heart catheterization;
Heart sound recorded with digital stethoscope;
13 without PH, 29 with PH;

### Porcine Dataset, PCG+SCG:

10 Pigs, each undergoing right heart catheterization;
Dataset size is 125 “pig patients” (by sampling sessions from the 10 pigs);
Each pig undergoes chemically induced hypertension multiple times. Heart sound is recorded at selected intervals;
Recording devices: Phonocardiography (PCG) and Seismocardiography (SCG);

### Human Dataset, SCG:

73 human subjects undergoing right heart catheterization and seismocardiography.

Each dataset was evaluated individually (via cross validation), and also evaluated for “cross domain generalization” (train one dataset and evaluate on the other). The method was evaluated with recordings of varying recording lengths.

TABLE 3
Varying recording length on Human (PCG) data

| | Macro | | Micro | |
|---|---|---|---|---|
| Recording Length | auROC | AP | auROC | AP |
| 5 min (baseline) | 0.94 | 0.98 | 0.92 | 0.96 |
| 20 heartbeats | 0.91 | 0.96 | 0.88 | 0.95 |
| 40 heartbeats | 0.91 | 0.97 | 0.90 | 0.95 |
| 80 heartbeats | 0.92 | 0.97 | 0.91 | 0.96 |
| 400 heartbeats | 0.91 | 0.97 | 0.88 | 0.95 |

Each number describes performance of 12 independently trained models, each undergoing 10-fold cross validation. Macro averages over each fold. Micro describes performance on each sample. auROC is area under the ROC curve. AP is average precision score (area under the PR curve).

TABLE 3
The test results for the method without the splitting step.
Baseline (cross validation)

| | Macro | | Micro | |
|---|---|---|---|---|
| Dataset | auROC | AP | auROC | AP |
| Human (PCG) | 0.94 | 0.98 | 0.92 | 0.96 |
| Porcine (PCG + SCG) | 0.92 | 0.94 | 0.81 | 0.82 |
| Human (SCG) | 0.75 | 0.92 | 0.73 | 0.90 |

Cross Domain Experiments (train → evaluate)

| | Macro | | Micro | |
|---|---|---|---|---|
| Dataset | auROC | AP | auROC | AP |
| Porcine → Human (PCG) | 0.82 (87%) | 0.92 (94%) | 0.82 (89%) | 0.92 (94%) |
| Human (SCG) → Human (PCG) | 0.80 (85%) | 0.91 (93%) | 0.78 (85%) | 0.89 (93%) |
| Human → Human (SCG) | 0.73 (97%) | 0.87 (95%) | 0.73 (100%) | 0.87 (97%) |
| Porcine → Human (SCG) | 0.59 (79%) | 0.83 (90%) | 0.59 (80%) | 0.83 (92%) |

Each number describes performance of 12 independently trained models, each undergoing 10-fold cross validation. Macro averages over each fold. Micro describes performance on each sample. auROC is area under the ROC curve. AP is average precision score (area under the PR curve). The model hyperparameters were tuned to the training partitions of the baseline Human (PCG) and Porcine (PCG+SCG) datasets. The percent number compares cross domain performance to baseline performance.

The results for the method with an extra step of splitting the splitting the sound signal (S2) into an aortic sound signal (A2) and a pulmonary sound signal (P2) and applying a pre-trained neural network to relate the generated one or more 2D feature maps with a training dataset of previously acquired, split, and generated training 2D, are similar, and slightly lower in some cases.

AP is Average Precision (area under precision-recall curve) and auROC is area under ROC curve. The two metrics together give a good sense of algorithm performance, including in the presence of class imbalance. Model hyperparameters are the same for all experiments.

Analysis of stethoscope heart sound data with deep networks is an effective, low-cost, and non-invasive solution for detection of pulmonary hypertension.

The present disclosure analyses heart sounds using deep networks, having low resource cost and is suitable for early screening.

The present disclosure comprises several advantages such as explanation of regions of interest in individual heartbeats and of all heartbeats overall and enhancing medical trustworthiness of model for particular subject prediction.

The term “comprising” whenever used in this document is intended to indicate the presence of stated features, integers, steps, components, but not to preclude the presence or addition of one or more other features, integers, steps, components, or groups thereof.

The disclosure should not be seen in any way restricted to the embodiments described and a person with ordinary skill in the art will foresee many possibilities to modifications thereof. The above-described embodiments are combinable.

The following claims further set out particular embodiments of the disclosure.

### REFERENCES

[1] Hasan B, Hansmann G, Budts W, Heath A, Hoodbhoy, Jing Z C, Koestenberger M, Meinel K, Mocumbi A O, Radchenko G D, et al. Challenges and special aspects of pulmonary hypertension in middle- to low-income regions: Jacc state-of-the-art review. Journal of the American College of Cardiology 2020; 75(19):2463-2477.
[2] Lau E M, Humbert M, Celermajer D S. Early detection of pulmonary arterial hypertension. Nature Reviews Cardiology 2015; 12(3):143-155.
[3] Taleb M, Khuder S, Tinkel J, Khouri S J. The diagnostic accuracy of d oppler echocardiography in assessment of pulmonary artery systolic pressure: A meta-analysis. Echocardiography 2013; 30(3):258-265.
[4] Oliveira J H, Renna F, Costa P, Nogueira D, Oliveira C, Fer258 reira C, Jorge A, Mattos S, Hatem T, Tavares T, Elola A, Rad A, Sameni R, Clifford G D, Coimbra M T. The circordigiscope dataset: From murmur detection to murmur classification. IEEE Journal of Biomedical and Health Informatics 2021; 1-1.
[5] Lang I M, Plank C, Sadushi-Kolici R, Jakowitsch J, Klepetko W, Maurer G. Imaging in pulmonary hyper tension. JACC Cardiovascular Imaging 2010; 3(12):1287-1295.
[6] Kaddoura T, Vadlamudi K, Kumar S, Bobhate P, Guo L, Jain S, Elgendi M, Coe J Y, Kim D, Taylor D, et al. Acoustic diagnosis of pulmonary hypertension: automated speech recognition-inspired classification algorithm outperforms physicians. scientific reports 2016; 6(1):1-11.
[7] Xu J, Durand L, Pibarot P. Nonlinear transient chirp signal modeling of the aortic and pulmonary components of the second heart sound. IEEE Transactions on Biomedical Engineering 2000; 47(10):1328-1335.
[8] Andreev V, Gramovich V, Krasikova M, Korolkov A, Vyborov O, Danilov N, Martynyuk T, Rodnenkov O, Rudenko O. Time-frequency analysis of the second heart sound to assess pulmonary artery pressure. Acoustical Physics 2020; 66(5):542-547.
[9] Dennis A, Michaels A D, Arand P, Ventura D. Noninvasive diagnosis of pulmonary hypertension using heart sound analysis. Computers in Biology and Medicine 2010; 40(9):758-764.
[10] Renna F, Oliveira J, Coimbra M T. Deep convolutional neural networks for heart sound segmentation. IEEE journal of biomedical and health informatics 2019; 23(6):2435-2445.
[11] Huang G, Liu Z, Van Der Maaten L, Weinberger K Q. Densely connected convolutional networks. In Proceedings of the IEEE conference on computer vision and pattern recognition. 2017; 4700-4708.
[12] Tan M, Le Q. Efficientnet: Rethinking model scaling for convolutional neural networks. In International conference on machine learning. PMLR, 2019; 6105-6114.
[13] Sundararajan M, Taly A, Yan Q. Axiomatic attribution for deep networks. In International conference on machine learning. PMLR, 2017; 3319-3328.
[14] Schmidt S E, Holst-Hansen C, Graff C, Toft E, Struijk J J. Segmentation of heart sound recordings by a duration dependent hidden markov model. Physiological measurement 2010; 31(4):513.
[15] Renna F, Plumbley M D, Coimbra M. Source separation of the second heart sound via alternating optimization. In 2021 Computing in Cardiology (CinC), volume 48. IEEE, 2021; 1-4.

1. A computer-implemented method for non-invasive estimation of Pulmonary Hypertension (“PH”), from heart sound signals, comprising the steps:
receiving a sound signal (S2) acquired from a beating heart of a subject over a predetermined time period;
generating one or more 2D feature maps comprising a 2D feature map with the received sound signal (S2), wherein a first axis of the map is arranged over time and a second axis of the map is arranged over individual heartbeats; and
applying a pre-trained neural network to relate the generated one or more 2D feature maps with a training dataset of previously acquired and generated training 2D feature maps of a PH subject group and a non-PH subject group to obtain an indicator of the presence of Pulmonary Hypertension.

2. The computer-implemented method for non-invasive estimation of Pulmonary Hypertension according to claim 1, further comprising the steps of:
splitting the sound signal (S2) into an aortic sound signal (A2) and a pulmonary sound signal (P2);
generating one or more 2D feature maps comprising a 2D pulmonary feature map with the pulmonary sound signal (P2), wherein a first axis of the map is arranged over time and a second axis of the map is arranged over individual heartbeats; and
applying a pre-trained neural network to relate the generated one or more 2D feature maps with a training dataset of previously acquired, split, and generated training 2D feature maps of a PH subject group and a non-PH subject group, to obtain an indicator of the presence of Pulmonary Hypertension.

3. The computer-implemented method for non-invasive estimation of Pulmonary Hypertension according to claim 2, wherein the one or more 2D feature maps comprising a 2D aortic feature map with the aortic sound signal (A2), wherein a first axis of the map is arranged over time and a second axis of the map is arranged over individual heartbeats.

4. The computer-implemented method for non-invasive estimation of Pulmonary Hypertension according to claim 2, wherein the one or more 2D feature maps comprise a 2D full-signal feature map with the received sound signal (S2), wherein a first axis of the map is arranged over time and a second axis of the map is arranged over individual heartbeats.

5. The computer-implemented method for non-invasive estimation of Pulmonary Hypertension according to claim 3, further comprising combining the one or more 2D feature maps as a multichannel input to the neural network, wherein each one or more 2D feature maps is combined as a channel of the multichannel input.

6. The computer-implemented method for non-invasive estimation of Pulmonary Hypertension according to claim 1, further comprising segmenting the acquired sound signal into a plurality of time windows of a predetermined duration, wherein each time window comprises a heartbeat sound signal peak.

7. The computer-implemented method for non-invasive estimation of Pulmonary Hypertension according to claim 6, further comprising aligning the segmented sound signal time windows by aligning the heartbeat sound signal peaks of the segmented sound signal time windows.

8. The computer-implemented method for non-invasive estimation of Pulmonary Hypertension according to claim 1, further comprising calculating a saliency attribution corresponding to the generated one or more 2D feature maps.

9. The computer-implemented method for non-invasive estimation of Pulmonary Hypertension according to claim 1, further comprising pre-processing the acquired sound signal by filtering, spike removal, normalizing, alignment, or segmentation, or a combination thereof.

10. The computer-implemented method for non-invasive estimation of Pulmonary Hypertension according to claim 1, wherein the neural network is selected from the group consisting of: a convolutional neural network (“CNN”), an over-parameterized deep neural network, and an extreme learning machine.

11. (canceled)

12. (canceled)

13. The computer-implemented method for non-invasive estimation of Pulmonary Hypertension according to claim 2, wherein the splitting is performed using an alternating optimization of a least-squares problem.

14. The computer-implemented method for non-invasive estimation of Pulmonary Hypertension according to claim 2, further comprising, after splitting the heart sound signal, filtering with second order Butterworth filters, and cleaning the heart sound signal by removing spikes.

15. The computer-implemented method for non-invasive estimation of Pulmonary Hypertension according to claim 1, further comprising acquiring the heart sound signal at the subject's pulmonary spot.

16. A computer-implemented method for training neural network for a non-invasive estimation of Pulmonary Hypertension (“PH”), from heart sound signals, comprising the steps, for both of a PH subject group and a non-PH subject group:
receiving a sound signal (S2) acquired from a beating heart of a subject over a predetermined time period;
generating one or more 2D feature maps comprising a 2D feature map with the received sound signal (S2), wherein a first axis of the map is arranged over time and a second axis of the map is arranged over individual heartbeats; and
applying a pre-trained neural network to relate the generated one or more 2D feature maps with a training dataset of previously acquired and generated training 2D feature maps of a PH subject group and a non-PH subject group to obtain an indicator of the presence of Pulmonary Hypertension.

17. The computer-implemented method for training neural network according to claim 16, further comprising the steps of:
splitting the heart sound (S2) signal into an aortic (A2) sound signal and a pulmonary (P2) sound signal;
generating one or more 2D feature maps comprising a 2D pulmonary feature map with the pulmonary sound signal (P2) where a first axis of the map is arranged over time and a second axis of the map is arranged over individual heartbeats; and
applying a pre-trained neural network to relate the generated one or more 2D feature maps with a training dataset of previously acquired, split and generated training 2D feature maps of a PH subject group and a non-PH subject group to obtain an indicator of the presence of Pulmonary Hypertension.

18. A computer-implemented system for non-invasive estimation of Pulmonary Hypertension (“PH”), from heart sound signals, comprising an electronic data processor arranged to carry out the steps:
receiving a sound signal (S2) acquired from a beating heart of a subject over a predetermined time period;
generating one or more 2D feature maps comprising a 2D feature map with the received sound signal (S2), wherein a first axis of the map is arranged over time and a second axis of the map is arranged over individual heartbeats; and
applying a pre-trained neural network to relate the generated one or more 2D feature maps with a training dataset of previously acquired, and generated training 2D feature maps of a PH subject group and a non-PH subject group to obtain an indicator of the presence of Pulmonary Hypertension.

19. The computer-implemented system according to claim 18, wherein the electronic data processor is further arranged to carry out the steps of:
splitting the sound signal (S2) into an aortic sound signal (A2) and a pulmonary sound signal (P2);
generating one or more 2D feature maps comprising a 2D pulmonary feature map with the pulmonary sound signal (P2) where a first axis of the map is arranged over time and a second axis of the map is arranged over individual heartbeats; and
applying a pre-trained neural network to relate the generated one or more 2D feature maps with a training dataset of previously acquired, split and generated training 2D feature maps of a PH subject group and a non-PH subject group, and to obtain an indicator of the presence of Pulmonary Hypertension.

20. The computer-implemented system according to claim 18, further comprising a digital stethoscope for acquiring the beating heart sound signal, wherein the digital stethoscope is connected to the electronic data processor and configured to transmit the acquired beating heart sound signal.

21. The computer-implemented system according to claim 18, wherein the electronic data processor is further arranged to segment the acquired sound signal into a plurality of time windows of a predetermined duration, wherein each time window comprises a heartbeat sound signal peak.

22. The computer-implemented system according to claim 18, wherein the electronic data processor is further arranged to align the segmented sound signal time windows by aligning the heartbeat sound signal peaks of the segmented sound signal time windows.
